(19) United States
(12) Patent Application Publication
(10) Pub. No.: US 2024/0082028 A1
(43) Pub. Date: Mar. 14, 2024

(54) SPIRALED PLASTIC PANCREATICOBILIARY STENT AND DEPLOYMENT SYSTEM

(71) Applicant: Regents of the University of Minnesota, Minneapolis, MN (US)

(72) Inventors: Stuart Kevin Amateau, Minneapolis, MN (US); Nicholas Michael McDonald, Minneapolis, MN (US)

(73) Assignee: Regents of the University of Minnesota, Minneapolis, MN (US)

(21) Appl. No.: 18/244,728

(22) Filed: Sep. 11, 2023

Related U.S. Application Data
(60) Provisional application No. 63/406,108, filed on Sep. 13, 2022.

(51) Int. Cl. A61F 2/94; A61F 2/966

(52) U.S. Cl. CPC A61F 2/94; A61F 2/966

(57) ABSTRACT

A stent and a system comprising a stent and a stent deployment catheter are disclosed. The stent comprises an elongated tubular body defining a longitudinal axis and including a proximal end, a distal end, and a central lumen extending between the proximal end and the distal end. The central lumen is configured for passage of a guide wire therethrough. The stent further comprises a tapered and/or frustoconical distal tip portion disposed at the distal end of the elongated tubular body, wherein the distal tip portion comprises one or more spiral threads. The stent deployment catheter comprises an inner sheath configured to be slidably disposed within the central lumen of the elongated tubular body; and an outer sheath configured to be slidably disposed over the inner sheath. The stent and stent deployment catheter can be configured for placement of the stent within a pancreaticobiliary duct.

### RELATED APPLICATIONS

This application claims priority to U.S. Provisional Application No. 63/406,108 that was filed on Sep. 13, 2022. The entire content of the application referenced above is hereby incorporated by reference herein.

### FIELD

The present application generally relates to medical devices and, in particular, to a ductal stent for traversing and treating pancreaticobiliary strictures.

### BACKGROUND

Placement of plastic pancreaticobiliary stents for drainage of the bile and pancreatic ducts is routinely performed in clinical practice. These stents are hollow tubes that are available in various configurations (e.g., straight, single pigtail, double pigtail), lengths, and diameters. They are used to traverse pancreaticobiliary strictures (duct narrowings) caused by a variety of etiologies, including stone-related disease, autoimmune disease, hereditary conditions, cancer, and postoperative complications. These stents are designed to maintain patency of stenotic ducts and to alleviate or prevent blockages or obstructions of bile and pancreatic juices.

Current methods for placement of pancreaticobiliary stents require a guidewire to first be threaded through the duct narrowing. The caliber and blunt end of the stent, in combination with the severity of the stricture, can make it difficult or impossible to get a stent across the stricture. There have been reports of using devices that are not intended for stent placement (e.g., a cytostome or stent retriever) to push through and/or dilate narrow strictures prior to attempting stent deployment. Such methods are often unsuccessful and have high risk of complication. Thus, there is a need for a device that provides alternative approaches for safe, efficient, and effective access through narrowed pancreaticobiliary strictures for stent deployment.

### BRIEF SUMMARY

A stent and a system comprising a stent and a stent deployment catheter are disclosed. The stent comprises an elongated tubular body defining a longitudinal axis and including a proximal end, a distal end, and a central lumen extending between the proximal end and the distal end. The central lumen is configured for passage of a guide wire therethrough. The stent further comprises a tapered and/or frustoconical distal tip portion disposed at the distal end of the elongated tubular body, wherein the distal tip portion comprises one or more spiral threads. The stent deployment catheter comprises an inner sheath configured to be slidably disposed within the central lumen of the elongated tubular body; and an outer sheath configured to be slidably disposed over the inner sheath. The stent and stent deployment catheter can be configured for placement of the stent within a pancreaticobiliary duct.

Accordingly, certain embodiments provide a stent comprising: an elongated tubular body defining a longitudinal axis and comprising a proximal end, a distal end, and a central lumen extending between the proximal end and the distal end, wherein the central lumen is configured for passage of a guide wire therethrough; and a tapered distal tip portion disposed at the distal end of the elongated tubular body, wherein the tapered distal tip portion comprises one or more spiral threads.

In certain embodiments, the elongated tubular body and the tapered distal tip portion comprise a biocompatible polymer.

In certain embodiments, the tapered distal tip portion is frustoconical in shape.

In certain embodiments, the one or more spiral threads is located on an outer surface of the tapered distal tip portion.

In certain embodiments, the tapered distal tip portion is configured for passage of a guide wire therethrough.

In certain embodiments, the stent further comprises at least one flexible retention flange configured to inhibit migration of the stent by atraumatically engaging tissue following insertion or implantation into a subject.

In certain embodiments, the at least one retention flange comprises a curved flat projection attached to an external surface of the elongated tubular body.

In certain embodiments, the at least one retention flange comprises a round disk surrounding an external surface of the elongated tubular body.

In certain embodiments, the elongated tubular body comprises at least one aperture configured to allow fluid communication between the central lumen and tissue proximate the stent.

In certain embodiments, at least a portion of the central lumen comprises a hexagonal cross section.

Certain embodiments provide a system comprising: 1) a stent comprising:
an elongated tubular body defining a longitudinal axis and comprising a proximal end, a distal end, and a central lumen extending between the proximal end and the distal end, wherein the central lumen is configured for passage of a guide wire therethrough; and a frustoconical distal tip portion disposed at the distal end of the elongated tubular body, wherein one or more spiral threads is attached to an outer surface of the frustoconical distal tip portion; and 2) a stent deployment catheter comprising: an inner sheath configured to be slidably disposed within the central lumen of the elongated tubular body; and an outer sheath configured to be slidably disposed over the inner sheath.

In certain embodiments, at least a portion of the inner sheath is configured to reversibly attach to or be fitted to the elongated tubular body of the stent, such that movement of the inner sheath results in corresponding movement of the elongated tubular body.

In certain embodiments, the movement comprises at least one of a turning, twisting, or rotating movement and a longitudinal pushing or pulling movement.

In certain embodiments, the frustoconical distal tip portion of the stent is configured to facilitate the movement.

In certain embodiments, the frustoconical distal tip portion of the stent is configured inhibit migration of the stent by atraumatically engaging tissue following insertion or implantation into a subject.

In certain embodiments, the central lumen of the elongated tubular body of the stent comprises an inner hexagonal circumference defining an inner diameter, wherein the inner sheath of the stent deployment catheter comprises an outer hexagonal circumference defining an outer diameter, and wherein the inner diameter of the inner hexagonal circumference of the central lumen is greater than the outer diameter of the outer hexagonal circumference of the inner sheath.

In certain embodiments, the proximal end of the elongated tubular body of the stent comprises a first outer diameter, wherein the outer sheath of the stent deployment catheter comprises a distal end having a second outer diameter, and wherein the first outer diameter and the second outer diameter are approximately equal.

In certain embodiments, the outer sheath of the stent deployment catheter is configured to push or advance the stent distally.

In certain embodiments, the elongated tubular body and the frustoconical distal tip portion of the stent comprise a biocompatible polymer.

In certain embodiments, the frustoconical distal tip portion of the stent is configured for passage of a guide wire therethrough.

In certain embodiments, the stent further comprises at least one flexible retention flange configured to inhibit migration of the stent by atraumatically engaging tissue following insertion or implantation into a subject.

In certain embodiments, the inner sheath and the outer sheath are configured to be removed once the stent is placed within an anatomical lumen of a subject.

In certain embodiments, the anatomical lumen is a pancreaticobiliary lumen.

Other objects, features, and advantages of the present invention will be apparent to one of skill in the art from the following detailed description and figures.

### BRIEF DESCRIPTION OF THE FIGURES

The present application can be understood by reference to the following drawings, wherein like reference numerals represent like elements. The drawings are merely exemplary to illustrate certain features that may be used singularly or in combination with other features and the present application should not be limited to the embodiments shown.

FIG. 1 is a side view of an embodiment of a stent in accordance with the present disclosure.

FIG. 2 is an enlarged side view of the proximal end portion of the stent of FIG. 1.

FIG. 3 is an enlarged side view of the distal end portion of the stent of FIG. 1.

FIG. 4 is a side view of an embodiment of a stent in accordance with the present disclosure.

FIG. 5 is an enlarged side view of the proximal end portion of the stent of FIG. 4.

FIG. 6 is a front, side perspective view of the proximal end portion of an embodiment of a stent in accordance with the present disclosure.

FIG. 7 is a front, top perspective view of the proximal end portion of the stent of FIG. 6.

FIG. 8 is a cross-sectional perspective view through line 8-8 shown in FIG. 1.

FIG. 9 is a cross-sectional perspective view through line 9-9 shown in FIG. 1.

FIG. 10 is an isometric cross-sectional view through line A-A shown in in FIG. 6.

FIG. 11 depicts schematic views of various embodiments of stents in accordance with the present disclosure.

FIG. 12A is a side view of an embodiment of an inner sheath of a stent deployment catheter in accordance with the present disclosure.

FIG. 12B is a front, side perspective view of the proximal end portion of the inner sheath shown in FIG. 12A.

FIG. 12C is a front, side perspective view of the distal end portion of the stent deployment catheter shown in FIG. 12A, including portions of both the inner sheath and the outer sheath.

FIG. 12D is a rear, side perspective view of the distal end portion of the stent deployment catheter shown in FIG. 12A, including portions of both the inner sheath and the outer sheath.

FIG. 12E is an isometric cross-sectional view through line B-B shown in FIG. 12D.

FIG. 13A is a side view of an embodiment of a stent positioned over an inner sheath of a stent deployment catheter in accordance with the present disclosure.

FIG. 13B is a rear, side perspective view of the stent and stent deployment catheter of FIG. 13A.

FIG. 13C is a front, side perspective view of the stent and stent deployment catheter of FIG. 13A.

FIG. 13D is a side view of an embodiment of a stent positioned over an inner sheath of a stent deployment catheter, which is positioned over a guidewire, in accordance with the present disclosure.

FIG. 13E is a rear, side perspective view of the stent and stent deployment catheter of FIG. 13D.

FIG. 13F is a front, side perspective view of the stent and stent deployment catheter of FIG. 13D.

FIG. 13G is a front, side perspective view of an embodiment of a stent positioned over an inner sheath of a stent deployment catheter in accordance with the present disclosure.

FIG. 13H is a rear, side perspective view of an embodiment of the stent positioned over the inner sheath of the stent deployment catheter of FIG. 13G.

### DETAILED DESCRIPTION

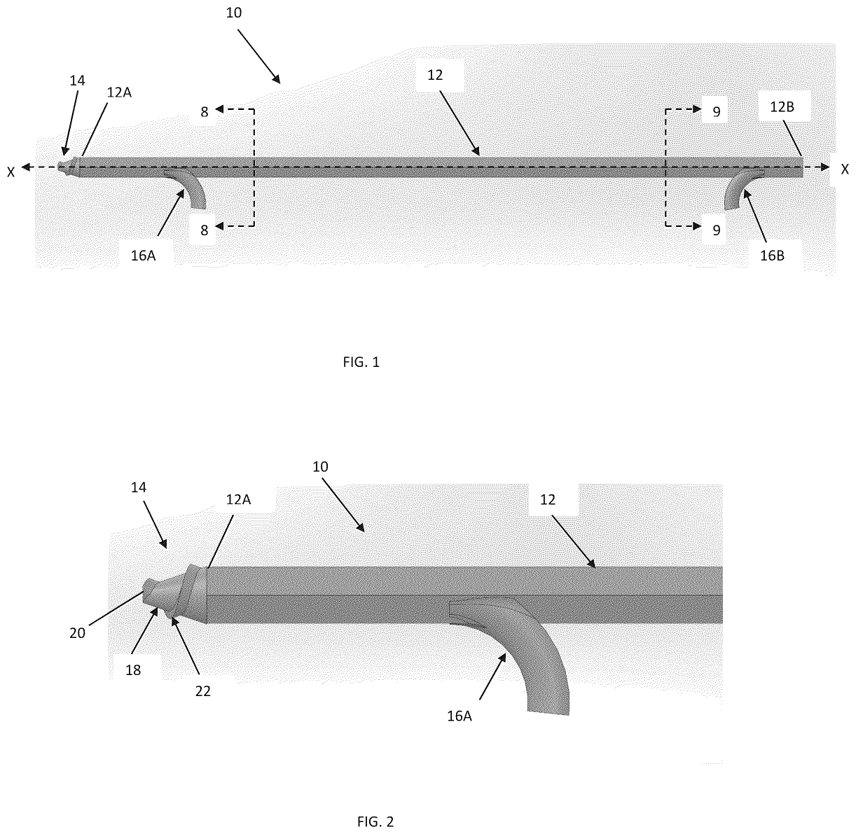

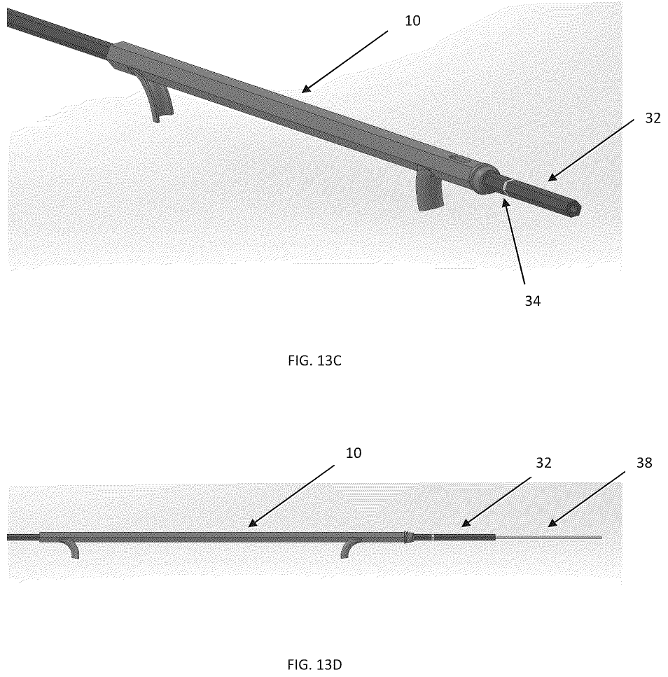

Referring now to the drawings wherein like reference numerals are used to identify like elements in the various views, FIG. 1 illustrates an exemplary embodiment of a stent 10 for use within a subject's body. As used herein, the term “subject” includes a vertebrate, such as a mammal, including but not limited to a human. The stent can comprise a flexible, biocompatible polymer, such as polyethylene, Teflon, Polytetrafluoroethylene (PTFE), silicon, polyurethane, or the like. Although the length of the stent 10 can vary, some exemplary lengths are shown in FIG. 11 (measurements in millimeters). The diameter of the stent 10 can also vary. For example, the stent diameter can be 5, 7, or 10 French. It should be noted that the stent 10 can be used in a variety of medical procedures, including both long- and short-wire endoscopic retrograde cholangiopancreatography (ERCP), as described, for example, in Reddy S C, Draganov P V, ERCP wire systems: The long and the short of it, World J Gastroenterol 2009 Jan. 7; 15(1): 55-60.

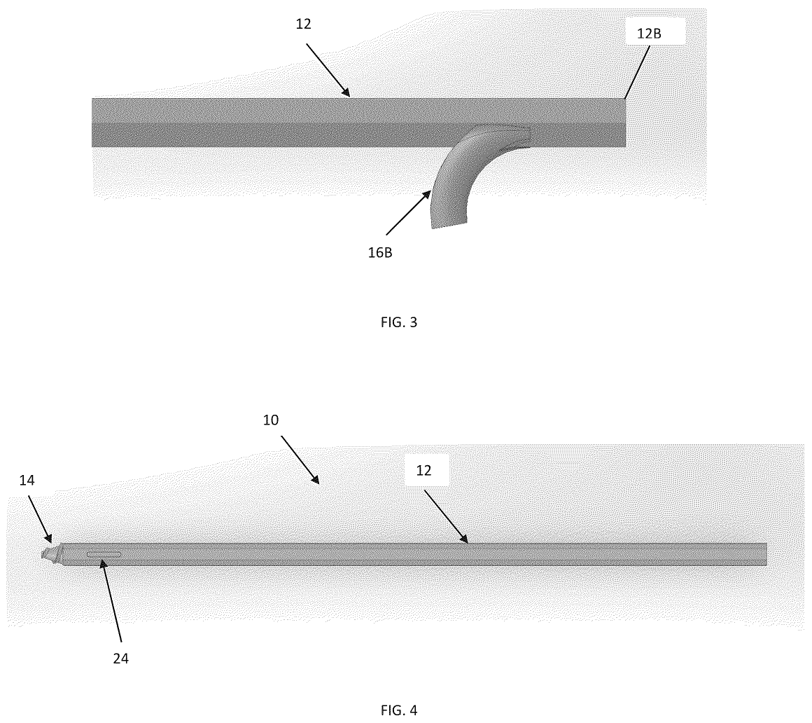

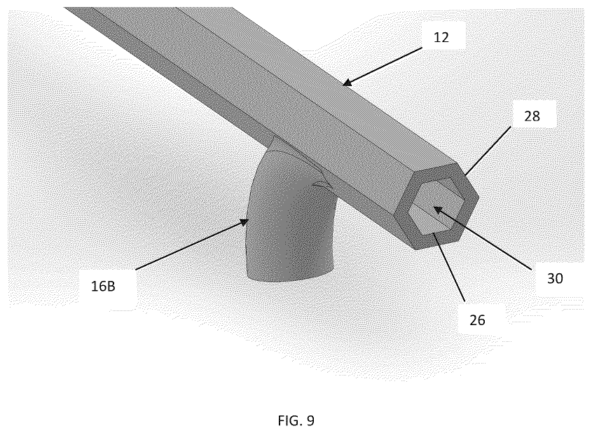

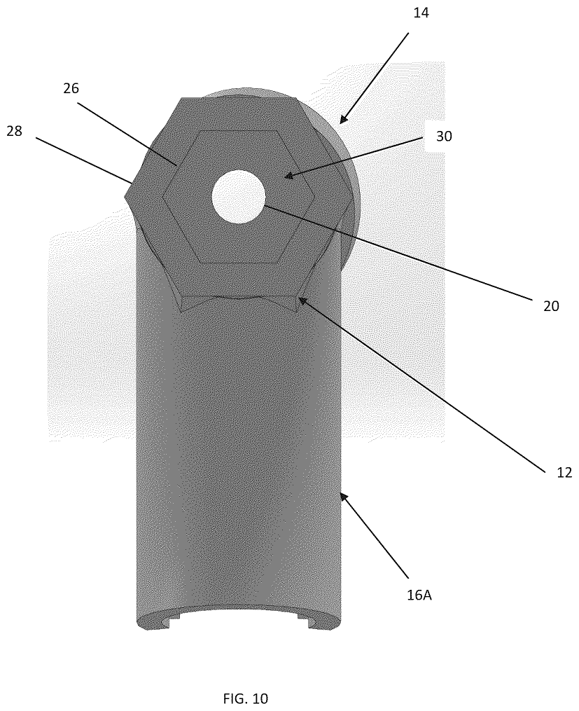

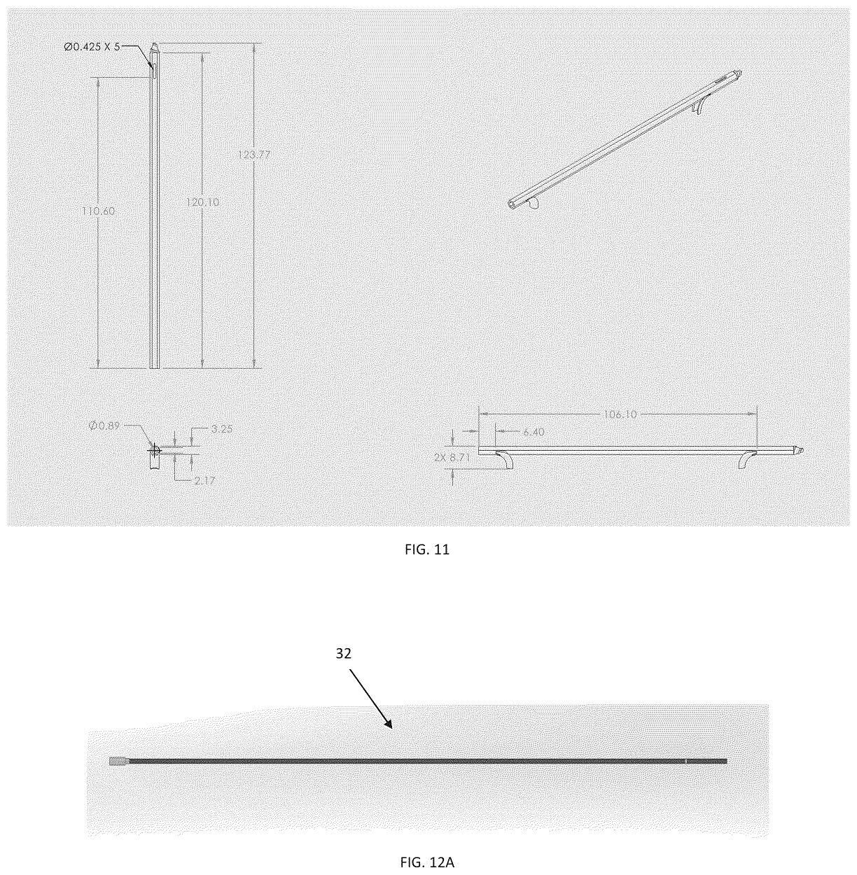

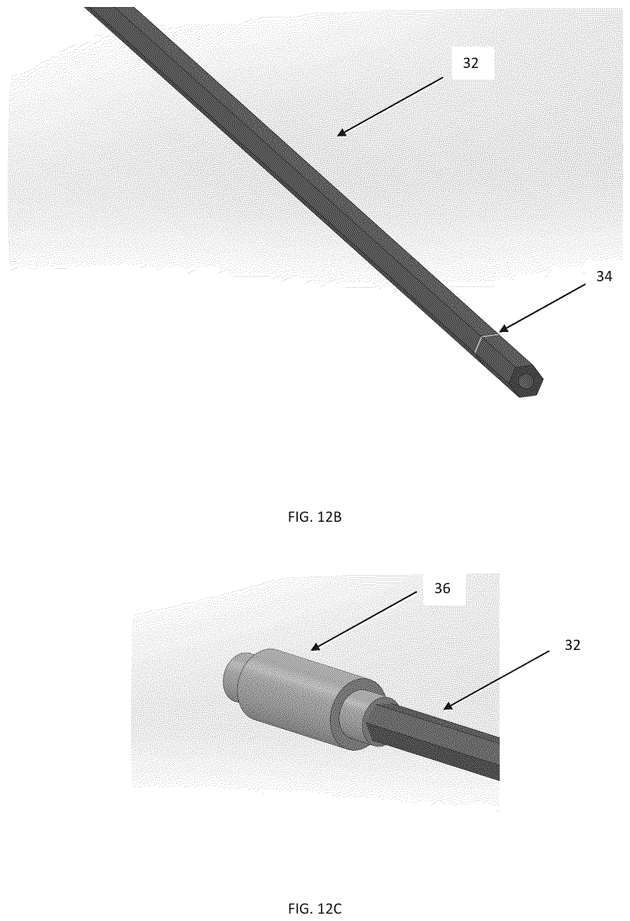

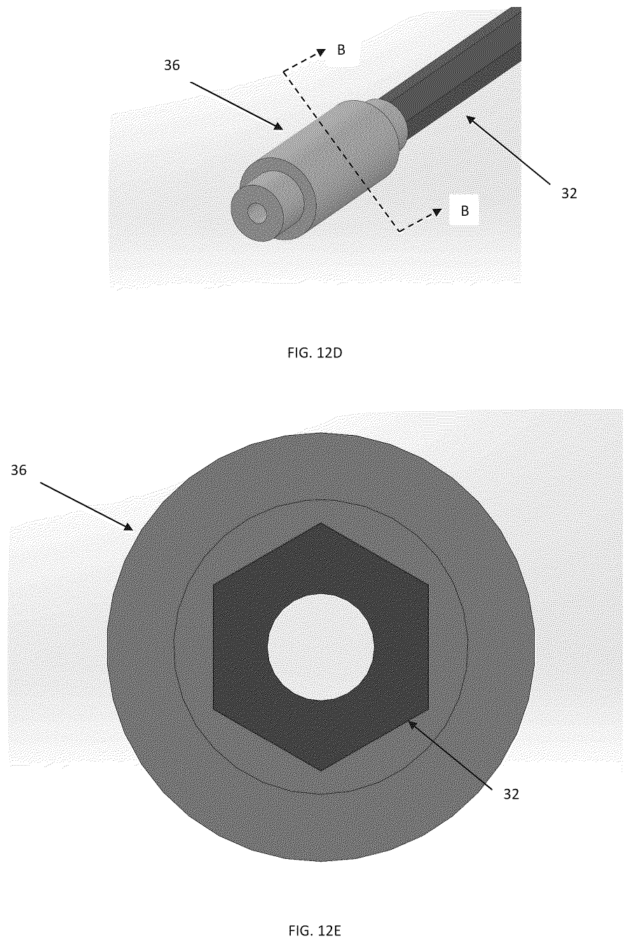

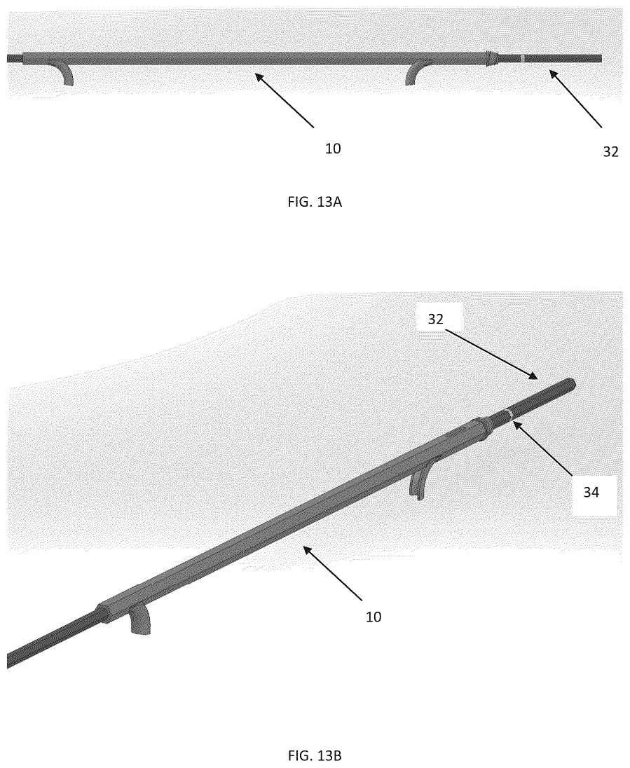

The stent 10 includes an elongated tubular body 12 with a central longitudinal axis X. The elongated tubular body 12 includes a proximal end 12A and a distal end 12B and a central lumen 30 (see, e.g., FIGS. 8-10) extending between the proximal end 12A and the distal end 12B. The central lumen 30 can be configured for passage of a guidewire (not shown) therethrough. A tapered distal tip portion 14 can be disposed at or near the proximal end 12A of the elongated tubular body 12. The tapered distal tip portion 14 can include a frustoconical tip 18 with a base lumen (not shown) and a blunt tip lumen 20, and one or more spiral threads 22 wound around the outer surface of the tip 18 (see, e.g., FIGS. 2 and 5-7). The tapered distal tip portion 14 can be configured such that a guidewire (and/or a deployment catheter, or a portion thereof) extending through the central lumen 30 of the elongated tubular body 12, can continue passage through the base lumen of the tip 18 and extend out from the blunt tip lumen 20. The one or more spiral threads 22 wound around the outer surface of the tip 18 can be ridged, as shown. The spiral threads 22 can be coarse or fine. The tapered distal tip portion 14 can be configured to allow the stent 10 to simultaneously twist, turn, or rotate while also advancing distally forward upon deployment within an anatomical duct. For example, the tapered distal tip portion 14 can allow the stent 10 to traverse narrowed or stenotic pancreaticobiliary strictures that would be inaccessible or difficult to traverse using a traditional plastic pancreaticobiliary stent. Moreover, the one or more spiral threads 22 can be configured to allow for optimal fit and secure placement of the stent 10 within narrowed passages by acting as a “screw” during stent deployment.

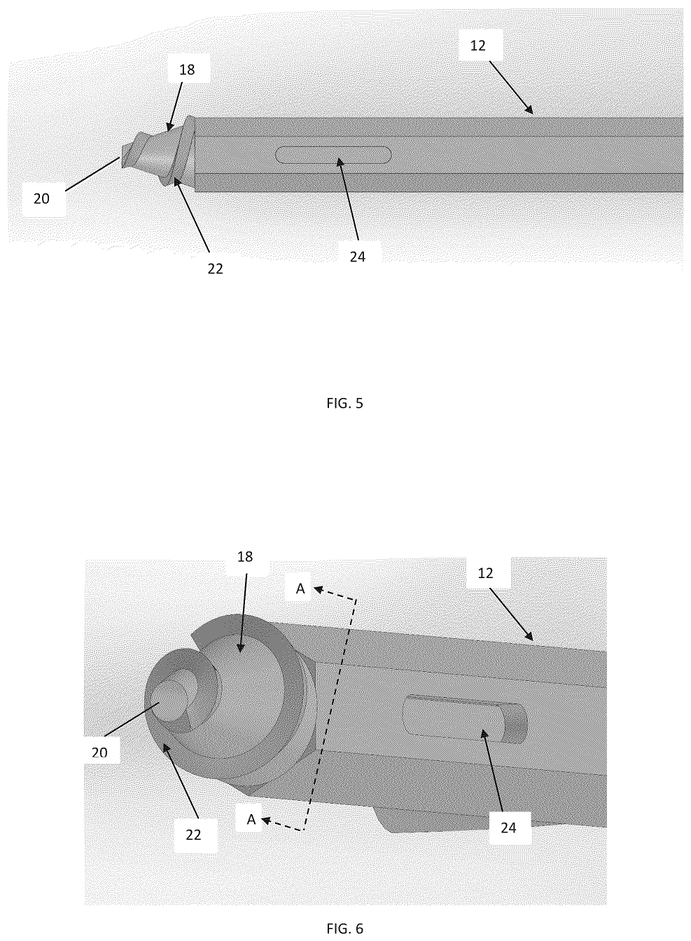

With continued reference to FIG. 1, the stent 10 can include one or more flexible retention flanges 16A, 16B configured to inhibit migration of the stent 10 by atraumatically engaging tissue following insertion or implantation into the subject. As shown in greater detail in FIGS. 2 and 3, the proximal retention flange 16A and the distal retention flange 16B can comprise a curved flat projection attached to the external surface of the elongated tubular body 12. In another embodiment, for example, the retention flanges 16A, 16B may comprise a round disk surrounding the external surface of the elongated tubular body 12.

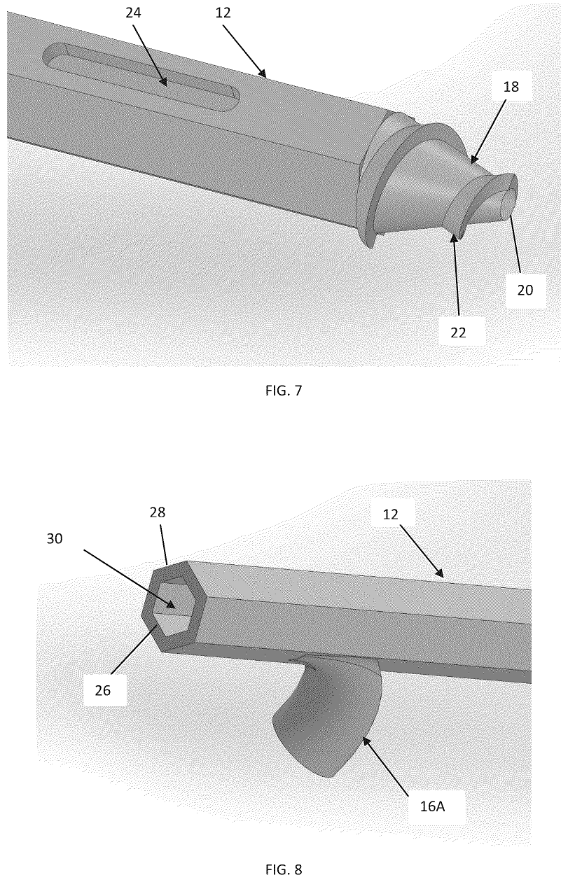

As shown in FIGS. 4-7, the elongated tubular body 12 can include at least one aperture 24 configured to allow fluid communication between the central lumen 30 and tissue proximate the stent 10. The aperture 24 can be oval shaped, as illustrated, or in can be configured in a variety of alternative shapes or forms. The aperture 24 can be located near the proximal end 12A of the elongated tubular body 12, as shown. Alternatively, the aperture 24 may be located near the distal end 12B or anywhere else along the outer surface of the elongated tubular body 12. The aperture 24 can allow for the passage of bodily fluids, such as bile or blood, for example, between the central lumen 30 of the stent 10 and the surrounding tissues.

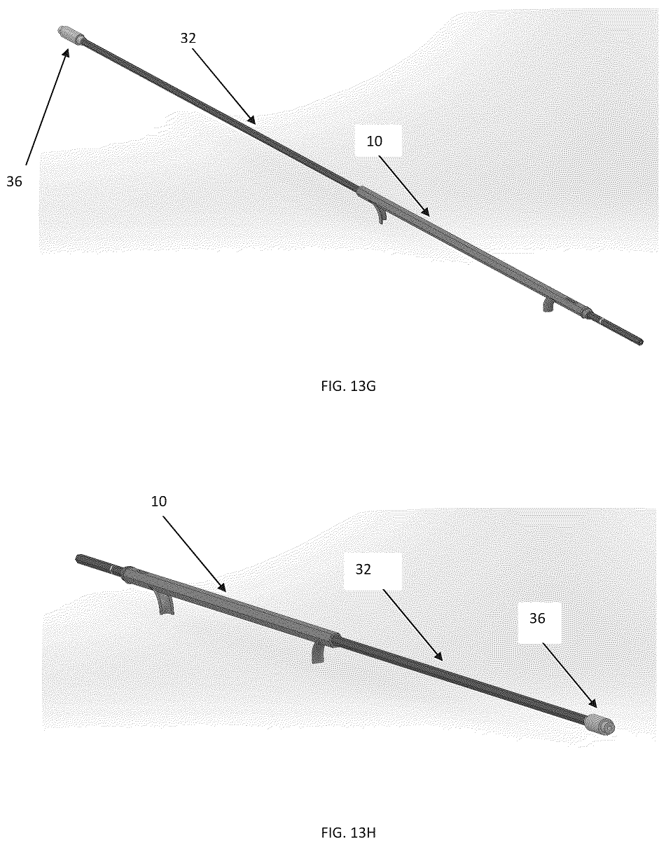

Referring now to FIGS. 8-10, the elongated tubular body 12 can have a variety of cross-sectional shapes. For example, in the embodiment shown, the elongated tubular body 12 has a hexagonal cross-section. In other embodiments, however, the cross-section of the elongated tubular body 12 may be circular, triangular, square, pentagonal, heptagonal, octagonal, nonagonal, decagonal, or any other two-dimensional shape. As shown in FIGS. 8-10, the central lumen 30 can also have a hexagonal cross-section. In other words, the elongated tubular body 12 can include a hexagonal inner circumference 26 defining the central lumen 30, as well as a hexagonal outer circumference 28. In other embodiments, however, the inner circumference 26 may be circular, triangular, square, pentagonal, heptagonal, octagonal, nonagonal, decagonal, or any other two-dimensional shape. In some embodiments, the inner circumference 26 and the outer circumference 28 may be different two-dimensional shapes. For example, the inner circumference 26 may be hexagonal and the outer circumference 28 may be circular. The shape of the inner circumference 26 can match the outer circumferential shape of a catheter or other deployment device configured to be inserted into the central lumen 30 (e.g., for stent placement). Consequently, the matching circumferential shapes can allow the stent and stent deployment device to lock or tightly fit together for secure engagement (see FIGS. 13A-13H).

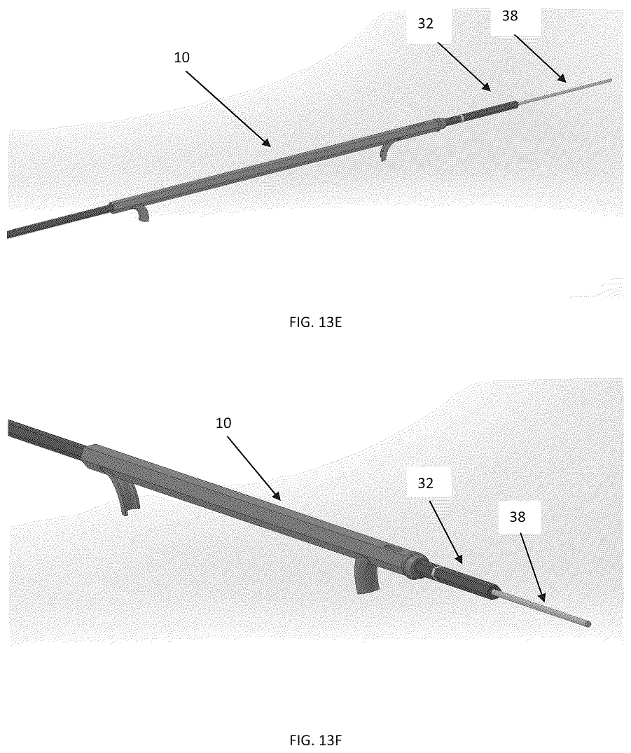

Referring now to FIGS. 12A-12E, an embodiment of an inner sheath 32 of a stent deployment catheter is shown. The inner sheath 32 can be configured to be slidably disposed over a guidewire 38, as shown in FIGS. 13D-13H. The inner sheath 32 can include a fluoroscopic band 34 at the distal end of the catheter for visualization when the catheter is being maneuvered within a subject's body. In some embodiments, the fluoroscopic band 34 may be located more proximally or more distally along the outer surface of the inner sheath 32. In some embodiments, there may be more than one fluoroscopic band 34. As shown in FIG. 12B, the inner sheath 32 can have a hexagonal outer circumference and a circular inner lumen. The hexagonal outer circumference of the inner sheath 32 allows it to be slidably disposed within the central lumen 30 of the elongated tubular body 12 of the stent 10, as the central lumen 30 has a hexagonal cross-section sized to tightly and securely fit around the inner sheath 32 (see FIGS. 8-10 and 13A-13H). In other embodiments, however, the inner sheath 32 may have an outer circumference and/or inner lumen of different circumferential shapes, including circular, triangular, square, pentagonal, heptagonal, octagonal, nonagonal, decagonal, or any other two-dimensional shape.

An embodiment of an outer sheath 36 of the stent deployment catheter is shown in FIGS. 12C-12E. The outer sheath 36 may be configured to be slidably disposed over the inner sheath 32. In the embodiment shown, the outer sheath 36 has a hexagonal inner lumen configured to fit over the hexagonal outer circumference of the inner sheath 32; however, the outer sheath 36 may have an outer circumference and/or inner lumen of different circumferential shapes, including circular, triangular, square, pentagonal, heptagonal, octagonal, nonagonal, decagonal, or any other two-dimensional shape. Moreover, the distal end of the outer sheath 36 may comprise a blunt end designed to abut the proximal end of the stent 10. The outer diameter of the proximal end of the stent 10 may be approximately equal to the outer diameter of the distal end of the outer sheath 36. As such, the outer sheath 36 may convey a lateral force along the central axis of the inner catheter 32, pushing the stent 10 distally. The outer sheath 36 (or “pusher member”) is further described in U.S. Patent Application No. 2005/0070794, which is hereby incorporated by reference in its entirety as if fully set forth herein.

As discussed above and further shown with respect to FIGS. 13A-13H, the central lumen 30 of the elongated tubular body 12 of the stent 10 is configured to be slidably disposed on and specifically fit to the outer surface of the inner sheath 32 of the deployment catheter (e.g., based on the shape and sizing of the stent and inner sheath of the catheter). In an embodiment, the stent 10 can be configured to reversibly lock (or be securely fitted) in place once positioned over the inner sheath 32. Consequently, movement of the inner sheath 32 results in corresponding movement of the stent 10. The movement of the stent 10 attached to the inner sheath 32 can be a turning, twisting, or rotating movement and/or a longitudinal pushing or pulling movement. The tapered distal tip portion 14 of the stent 10 can facilitate such movement, aided by the one or more spiral threads 22 wound around the outer surface of the tip 18 (see, e.g., FIGS. 2 and 5-7). As a result, the stent 10 can more easily and effectively traverse narrowed strictures (e.g., pancreaticobiliary strictures) during placement. Following placement, the tapered distal tip portion 14 can also serve to inhibit migration of the stent 10 by atraumatically engaging surrounding tissue. Thus, the stent 10 is configured for both improved initial access and long-term, stable placement following deployment within an anatomical duct.

Although at least one embodiment of a pancreaticobiliary stent and deployment system has described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and can include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure can be made without departing from the spirit of the disclosure as defined in the appended claims.

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A stent comprising:
an elongated tubular body defining a longitudinal axis and comprising a proximal end, a distal end, and a central lumen extending between the proximal end and the distal end, wherein the central lumen is configured for passage of a guide wire therethrough; and
a tapered distal tip portion disposed at the distal end of the elongated tubular body, wherein the tapered distal tip portion comprises one or more spiral threads.

2. The stent of claim 1, wherein the elongated tubular body and the tapered distal tip portion comprise a biocompatible polymer.

3. The stent of claim 1, wherein the tapered distal tip portion is frustoconical in shape.

4. The stent of claim 1, wherein the one or more spiral threads is located on an outer surface of the tapered distal tip portion.

5. The stent of claim 1, wherein the tapered distal tip portion is configured for passage of a guide wire therethrough.

6. The stent of claim 1, further comprising at least one flexible retention flange configured to inhibit migration of the stent by atraumatically engaging tissue following insertion or implantation into a subject.

7. The stent of claim 6, wherein the at least one retention flange comprises a curved flat projection attached to an external surface of the elongated tubular body.

8. The stent of claim 6, wherein the at least one retention flange comprises a round disk surrounding an external surface of the elongated tubular body.

9. The stent of claim 1, wherein the elongated tubular body comprises at least one aperture configured to allow fluid communication between the central lumen and tissue proximate the stent.

10. The stent of claim 1, wherein at least a portion of the central lumen comprises a hexagonal cross section.

11. A system comprising:
a stent comprising:
an elongated tubular body defining a longitudinal axis and comprising a proximal end, a distal end, and a central lumen extending between the proximal end and the distal end, wherein the central lumen is configured for passage of a guide wire therethrough; and
a frustoconical distal tip portion disposed at the distal end of the elongated tubular body, wherein one or more spiral threads is attached to an outer surface of the frustoconical distal tip portion; and
a stent deployment catheter comprising:
an inner sheath configured to be slidably disposed within the central lumen of the elongated tubular body; and
an outer sheath configured to be slidably disposed over the inner sheath.

12. The system of claim 11, wherein at least a portion of the inner sheath is configured to reversibly attach to or be fitted to the elongated tubular body of the stent, such that movement of the inner sheath results in corresponding movement of the elongated tubular body.

13. The system of claim 12, wherein the movement comprises at least one of a turning, twisting, or rotating movement and a longitudinal pushing or pulling movement.

14. The system of claim 13, wherein the frustoconical distal tip portion of the stent is configured to facilitate the movement.

15. The system of claim 11, wherein the frustoconical distal tip portion of the stent is configured inhibit migration of the stent by atraumatically engaging tissue following insertion or implantation into a subject.

16. The system of claim 11, wherein the central lumen of the elongated tubular body of the stent comprises an inner hexagonal circumference defining an inner diameter, wherein the inner sheath of the stent deployment catheter comprises an outer hexagonal circumference defining an outer diameter, and wherein the inner diameter of the inner hexagonal circumference of the central lumen is greater than the outer diameter of the outer hexagonal circumference of the inner sheath.

17. The system of claim 11, wherein the proximal end of the elongated tubular body of the stent comprises a first outer diameter, wherein the outer sheath of the stent deployment catheter comprises a distal end having a second outer diameter, and wherein the first outer diameter and the second outer diameter are approximately equal.

18. The system of claim 11, wherein the outer sheath of the stent deployment catheter is configured to push or advance the stent distally.

19. The system of claim 11, wherein the elongated tubular body and the frustoconical distal tip portion of the stent comprise a biocompatible polymer.

20. The system of claim 11, wherein the frustoconical distal tip portion of the stent is configured for passage of a guide wire therethrough.

21. The system of claim 11, wherein the stent further comprises at least one flexible retention flange configured to inhibit migration of the stent by atraumatically engaging tissue following insertion or implantation into a subject.

22. The system of claim 11, wherein the inner sheath and the outer sheath are configured to be removed once the stent is placed within an anatomical lumen of a subject.

23. The system of claim 22, wherein the anatomical lumen is a pancreaticobiliary lumen.
